(19) United States
(12) Patent Application Publication
(10) Pub. No.: US 2007/0178047 A1
(43) Pub. Date: Aug. 2, 2007

(54) Drug carrier and ultrasound apparatus

(76) Inventor: Kenichi Kawabata, Kodaira (JP)

Correspondence Address: MATTINGLY, STANGER, MALUR & BRUNDIDGE, P.C., 1800 DIAGONAL ROAD, SUITE 370, ALEXANDRIA, VA, 22314, US

(21) Appl. No.: 11/653,948

(22) Filed: Jan. 17, 2007

(30) Foreign Application Priority Data
Jan. 30, 2006 (JP) 2006-020495

(51) Int. Cl. A61K 49/22; A61B 8/00

(52) U.S. Cl. 424/9.5; 600/437

(57) ABSTRACT

A drug carrier and an ultrasound apparatus used in combination therewith for releasing a drug. The drug carrier undergoes a reversible phase change from liquid to gas upon ultrasound irradiation, so that the presence of the drug can be detected with a diagnostic apparatus without causing the spilling of the encased drug. The drug carrier includes a drug that is contained in a mixture of a poorly water-soluble substance having a boiling point of 37° C. or lower and a poorly water-soluble substance having a boiling point of higher than 37° C., which mixture is further encapsulated by a membrane of amphipathic substance.

### CLAIM OF PRIORITY

The present application claims priority from Japanese application JP 2006-020495 filed on Jan. 30, 2006, the contents of which are hereby incorporated by reference into this application.

### BACKGROUND OF THE INVENTION

1. Field of the Invention

The present invention relates to a carrier for delivering drug to an affected area. It also relates to a medical ultrasound apparatus for releasing the drug from the carrier by ultrasound irradiation.

2. Background Art

When it is desired to use a drug as a sustained-release drug that stays inside the body for a long time, or when a high drug concentration is desired at a target site alone, a drug delivery system (DDS) is often used whereby a drug is internally administered by encapsulating it in a carrier composed of a surfactant, phospholipid, protein, or the like, rather than administering it as is. Such DDS's include a passive system based on the gradual leakage of drug from the carrier with the passage of time, and an active system in which the carrier is destructed by an external stimulus so as to increase the drug concentration at a specific site in an active manner. In the latter, active DDS system, the most widely used external stimulus is temperature. Phospholipid transitions from a gel state to a liquid crystal state above a certain temperature called phase transition temperature, resulting in higher fluidity. By encapsulating the drug in a phospholipid membrane whose phase transition temperature is set to be slightly higher than the body temperature, only those parts of the internally administered drug that reached the target site are released from the carrier phospholipid membrane upon heating.

Such technique whereby the permeability of the carrier is increased by temperature rise is problematic in the following two respects:

### (1) Release of drug takes time because the technique merely involves an increase in the permeability of carrier membrane, rather than instantaneous destruction of the membrane.

### (2) It is difficult to achieve a localized increase in temperature inside the body with such a weak level of heating (42 C° or lower) that it will not affect normal tissues, because body temperature is equalized by blood flows.

In contrast to such DDS's utilizing increases in temperature, there is another technique whereby the carrier is destructed by ultrasound energy. The technique is based on the phenomenon in which micrometer-size bubbles resonate with ultrasound waves of frequencies around several MHz that are used for diagnostic purposes. The bubbles are stabilized with phospholipid or the like, and the drug is encapsulated in a phospholipid membrane. As the bubbles are destructed, the drug is released. As compared with the foregoing method utilizing temperature rise, this method employing ultrasound energy is advantageous in the following two points:

### (1) Because the carrier is destructed, the drug can be released instantaneously.

### (2) Ultrasound energy can be localized within a very small area of 1 cm³ or less using a converging wave.

Non-patent Document 1: Allen, Nature Rev. Cancer 2:750-763 (2002)

Non-patent Document 2: Winter et al., Magnetic Resonance in Medicine 50:411-416 (2003)

Non-patent Document 3: Grant et al., Magnetic Resonance in Medicine 11:236-243 (1989)

Non-patent Document 4: Sahoo et al., Langmuir 17:7907-7911 (2001)

### SUMMARY OF THE INVENTION

The above method employing ultrasound energy has the following two disadvantages:

### (1) The gas is easily exhausted from the lungs and stays inside the body only for a short time.

(2) While the contrast-agent function of the bubbles allows to check whether or not the drug is present at the target site, the bubbles are destructed upon checking, making it impossible to obtain feedback concerning the concentration of the drug, for example. Namely, the ultrasound contrast-agent function cannot be utilized.

The first disadvantage can be overcome by, e.g., using a phase-change type carrier that is liquid upon administration but is rendered into microbubbles by ultrasound irradiation, instead of directly administering microbubbles. However, it has been unable to overcome the second disadvantage with the conventional drug or ultrasound systems.

Thus, the conventional DDS based on the direct application of ultrasound energy, while capable of instantaneous release of drug by the destruction of the bubbles, has been unable to take advantage of the contrast-agent effect of the bubbles and to release drug at an appropriate timing, due to the contrast-agent function and the release of the drug occurring simultaneously.

The invention is based on the inventors' realization that the aforementioned problems can be solved by using a drug carrier that is liquid and has the function of a drug carrier upon administration into a living organism, that forms into bubbles upon ultrasound irradiation, and that returns to the original liquid upon termination of ultrasound irradiation. Normally, the principal component of a phase-change type ultrasound contrast agent that turns from liquid into gas upon ultrasound irradiation is a volatile poorly water-soluble substance having a boiling point of 37° C. or lower, such as perfluoropentane. Such substance, if internally administered as is, would be immediately boiled inside the body. However, if it is rendered into fine particles by emulsification, for example, its apparent boiling point increases due to the fact that the interfacial tension is inversely proportional to the radius of the liquid fine particle. As a result, the substance is readily vaporized upon internal administration. If this is followed by ultrasound irradiation, the emulsion system would be destroyed and the poorly water-soluble substance would be in a close-to-naked state, resulting in vaporization at temperature exceeding the boiling point. Thus, when a volatile and poorly water-soluble substance of 37° C. or lower is used, the bubbles produced by vaporization of a liquid exist irreversibly and do not return to liquid.

The inventors have discovered a phenomenon in which a poorly water-soluble substance having a boiling point with a boiling point of 37° C. or higher is turned from liquid into gas upon ultrasound irradiation, and in which the gas turns back into liquid upon termination of ultrasound irradiation. However, to vaporize a poorly water-soluble substance having a boiling point of 37° C. or higher normally requires ultrasound irradiation of high intensity, with the potential increase in invasiveness. The inventors' further analysis led to the following discovery. That is, when a mixture solution of a poorly water-soluble substance having the boiling point of more than 37° C. (high-boiling point substance) and a poorly water-soluble substance having the boiling point of 37° C. or lower (low-boiling point substance) is used, if the high-boiling point substance and the low-boiling point substance have similar structures, i.e., if they are both fluorocarbons, hydrocarbons, or if the other is a substitution of several fluorine atoms of one substance with hydrogen, they interact with each other, resulting in the vaporization of the low-boiling point substance first upon ultrasound irradiation. The vaporization is accompanied by an increase in the ultrasound absorption coefficient of the mixture, resulting in the secondary vaporization of the high boiling-point compound. Thus, a carrier can be realized that can be reversibly turned from liquid into gas by low-intensity ultrasound irradiation of 10 W/cm² or less. Particularly, it was found that stability could be increased by using a high boiling-point compound of fluorocarbon or fluorohydrocarbon having the boiling point of 60° C. or higher and 100° C. or lower.

The carrier is desirably in the form of micelle, emulsion, or liposome having a highly biocompatible phospholipid as a principal component; the form, however, is not particularly limited as long as it does not interfere with ultrasound phase-change. The form of the carrier when encapsulating a drug may also vary depending on whether the drug is water-soluble or lipophilic. FIGS. 1A to 1C show structures of the carrier when encapsulating a drug.

FIGS. 1A and 1B show structures of the carrier encapsulating a water-soluble drug and a lipophilic drug, respectively. FIG. 1C shows a structure in a case where the carrier encapsulates a water-soluble drug in the form of a reversed micelle. In FIGS. 1A to 1C, the oil phase is a phase that includes a poorly water-soluble substance alone that undergoes phase-change upon ultrasound irradiation, and a mixture of such poorly water-soluble substance and an oil that is highly biocompatible, such as vegetable oil. The aqueous phase consists of an isotonic solution that can be administered to living organisms, such as normal saline or a phosphate buffer. The surfactant phase includes, in FIGS. 1A and 1B, both a highly biocompatible surfactant containing phospholipid alone, and a mixture of such surfactant and a stabilizing component. The carrier shown in FIG. 1A consists of an aqueous phase containing a drug that is covered with a surfactant phase, on the outside of which there is further an oil phase that is covered with a surfactant phase. The carrier shown in FIG. 1B consists of an oil phase containing a drug that is covered with a surfactant phase. The carrier shown in FIG. 1C consists of an oil phase containing a drug that is covered with a surfactant phase, wherein the oil phase is covered with another surfactant phase.

The drug carrier of the invention includes a poorly water-soluble compound having a boiling point of 37° C. or lower (compound 1) and a poorly water-soluble compound (compound 2) having a boiling point of higher than 37° C. Preferably, compound 1 and compound 2 have a molar ratio of 0.1 or higher and 4 or lower. The carrier of the invention may have a membrane structure containing compound 1 and compound 2, the membrane being made of an amphipathic substance, such as phospholipid, surfactant, or protein. The membrane structure may be in the form of micelle, emulsion, or liposome. The carrier of the invention may include a water-soluble drug dispersed, in the form of a reversed micelle using a fluorine surfactant, in a physiologically permissible organic solvent, such as vegetable oil. The drug observation/releasing device of the invention has an observation mode for the measurement of the degree of accumulation of the drug at a target site by reversibly turning the carrier from liquid into gas, and a destruction mode for irreversibly turning the carrier from liquid into gas in order to release the drug. The device may be configured such that, after being turned on and before going into the destruction mode, it is determined whether or not the observation mode has been activated at least once and, if not, the destruction mode is prohibited.

In accordance with the invention, a phase change from liquid into gas can be reversibly caused without spilling the drug, so that the presence of the drug carrier can be confirmed. Furthermore, the drug carrier can be irreversibly destructed after confirming that the carrier including the drug is in an appropriate condition, so that the drug can be irreversibly released. These features provide a safe diagnostic and therapeutic technique.

### BRIEF DESCRIPTION OF THE DRAWINGS

FIGS. 1A to 1C show conceptual charts illustrating the structure of the drug carrier according to the invention.

FIG. 2 shows the configuration of an experiment system for testing the effect of the drug carrier of the invention.

FIG. 3 shows an example of a test demonstrating reversibility concerning ultrasound irradiation of the drug carrier of the invention.

FIG. 4 shows an example of a test demonstrating reversibility concerning ultrasound irradiation of the drug carrier of the invention.

FIG. 5 shows an example of a test demonstrating the effect of a composition of the drug carrier of the invention on reversibility concerning ultrasound irradiation.

FIG. 6 shows an example of a test demonstrating irreversibility concerning ultrasound irradiation of the drug carrier of the invention.

FIG. 7 shows the configuration of an embodiment of the drug-releasing ultrasound apparatus of the invention.

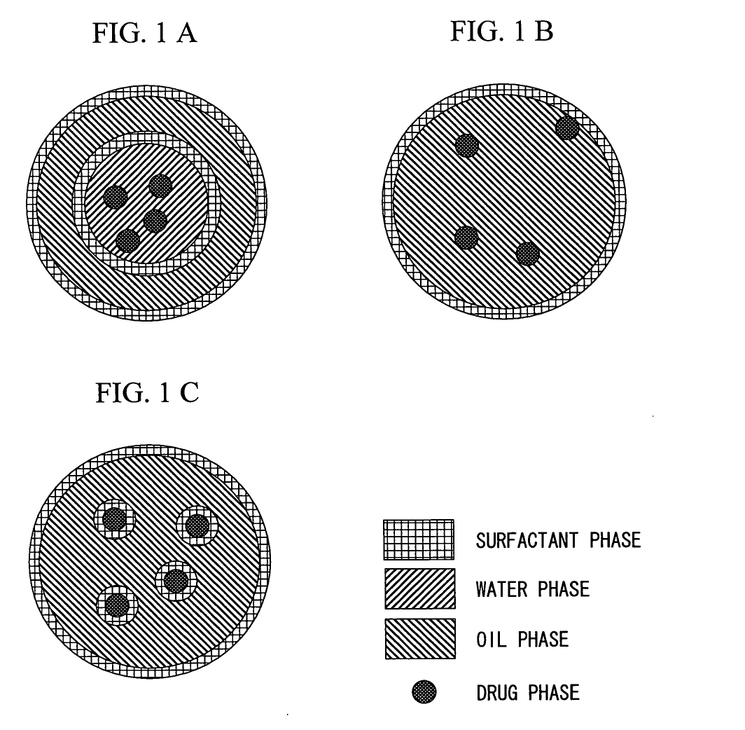

### DESCRIPTION OF THE PREFERRED EMBODIMENTS

In the following, tests conducted to demonstrate the validity of the carriers according to the invention, and examples of the invention are described. The invention, however, is not limited to such examples.

### (Test 1) Test Concerning the Reversibility of the Contrast-Agent Effect

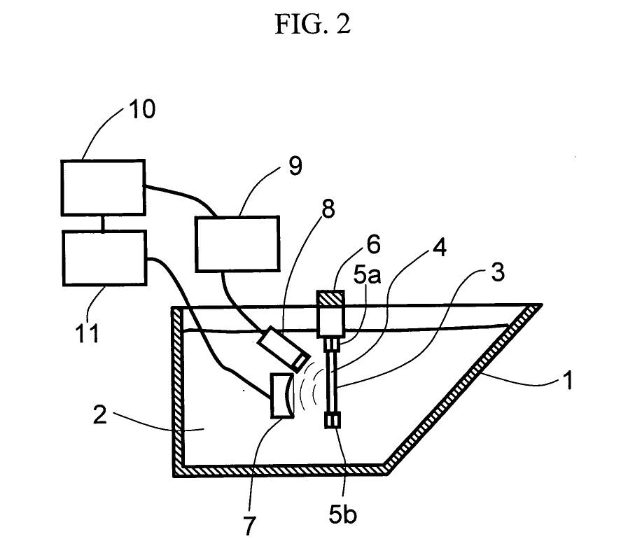

In order to show that the drug carrier of the invention reversibly turns from liquid into gas upon ultrasound irradiation, a test was conducted, which will be described with reference to FIGS. 2, 3, and 4.

FIG. 2 shows an experiment system for the test. This experiment system includes a resin-made water bath 1, a degassed water 2 set at 37° C., sample encapsulating tube 3, a sample 4, tube-end fixing clips 5a and 5b, a sample fixture 6, a transducer 7 for generating a focused ultrasound wave for sample-phase change, an ultrasound diagnostic apparatus probe 8 for phase-change observation, an ultrasound diagnostic apparatus 9, a phase-change ultrasound signal generating apparatus 10, and an amplifier 11. The test was conducted in the following way. First, a carrier was prepared by the following technique. The components indicated below were added together, and normal saline was added slowly until the total volume became 25 ml. The mixture was then homogenized with ULTRA-TURRAX T25 (Janke&Knukel, Staufen, Germany) at 9500 rpm for one minute at ice temperature.

| | | |
|---|---|---|
| glycerol | 2.0 | g |
| α-tocopherol | 0.02 | g |
| cholesterol | 0.1 | g |
| lecithin | 1.0 | g |
| perfluoropentane | 0.086 | g (300 nmol) |
| perfluoroheptane | 0.27 | g (700 nmol) |

The emulsion was subjected to high-pressure emulsification using Emulsiflex-C5 (Avestin, Ottawa, Canada) at 20 MPa for 2 minutes, and then filtered by a 0.4-μm membrane filter. These processes yielded a substantially transparent microemulsion, of which 98% or more had diameters of 200 nm or smaller as measured with LB-550 (Horiba, Ltd., Tokyo).

Then, using the experiment system shown in FIG. 2, the prepared carrier was encapsulated in the sample encapsulating tube 3 (Tygon® tube having an inner diameter of 1.59 mm and an outer diameter of 3.18 mm). While observing with the phase-change observation ultrasound diagnostic apparatus probe (Hitachi Medical Corp., EUP-L53S, 7.5 MHz) 8 and the ultrasound diagnostic apparatus (Hitachi Medical Corp., EUB-8500) 9, the carrier was irradiated with pulsed ultrasound emitted by a transducer 7 for generating a focused ultrasound wave for phase change of the sample (frequency: 3.4 MHz, diameter: 40 mm, F number: 1). On the ultrasound diagnostic apparatus 9, ultrasound diagnostic apparatus images were acquired before, during, and after the ultrasound irradiation by the transducer 7. The transducer 7 and the ultrasound diagnostic apparatus 9 were synchronized, such that when the sample was being hit by the transmission/reception waves of the diagnostic ultrasound emitted by the phase-change observation ultrasound diagnostic apparatus probe 8, no ultrasound was emitted by the transducer 7 for generating a focused ultrasound wave for phase change of samples.

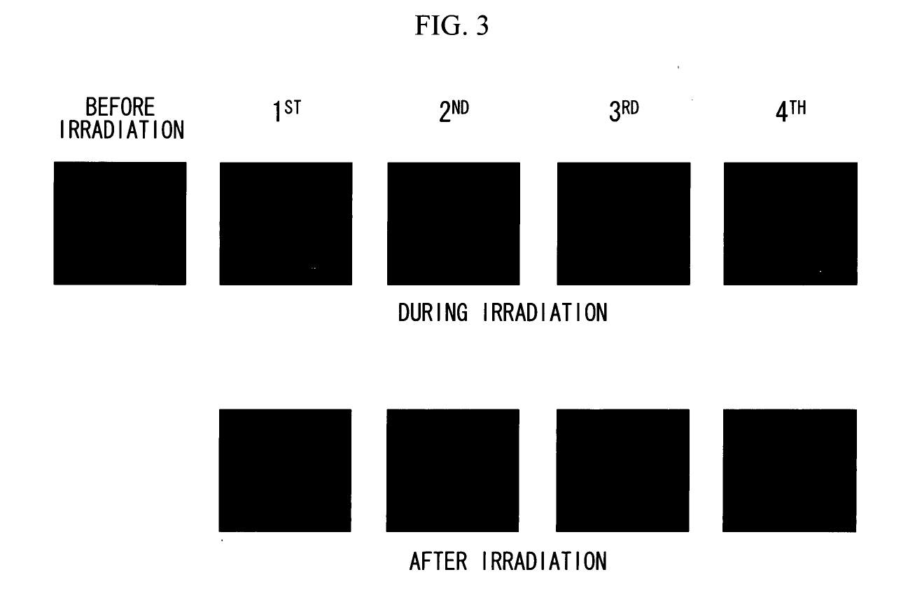

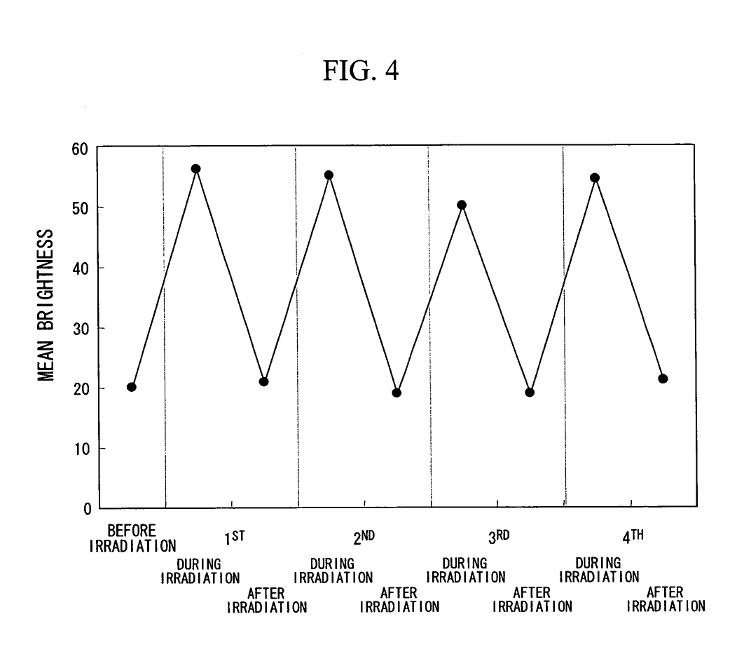

An example of the obtained results is described with reference to FIGS. 3 and 4. FIG. 3 shows ultrasound tomographic images of the sample upon irradiation with ultrasound emitted by the transducer 7 for generating a focused ultrasound wave for sample-phase change 7, having the frequency of 3.4 MHz, and peak intensity of 4 W/cm², pulse period of 35 ms (5 ms on, 30 ms off), for 0.5 second. The images were obtained with the ultrasound diagnostic apparatus 9 in the WPI mode. The ultrasound irradiation by the transducer 7 for generating a focused ultrasound wave for sample-phase change was conducted four times. These images are those obtained during and after the ultrasound irradiation. In all of the four instances of irradiation, the brightness of the sample increased during ultrasound irradiation and then returned back after irradiation.

FIG. 4 shows a numerical representation of the mean brightness of the sample based on the ultrasound tomographic images shown in FIG. 3. It can be seen from FIGS. 3 and 4 that the drug carrier of the invention underwent a change in its brightness reversibly upon ultrasound irradiation. Since the WPI mode is a rendering mode that is particularly sensitive to microbubbles, it is clear that the drug carrier of the invention causes reversible phase-change between liquid and gas upon ultrasound irradiation. Similar results were obtained when another test was conducted in which the ultrasound peak intensity was varied in the range of 1 to 20 W/cm². Substantially similar results were also obtained in a test in which the frequency of the ultrasound emitted by the transducer 7 for generating a focused ultrasound wave for sample-phase change was varied in the range of 0.5 to 7 MHz. The results were also substantially the same when a test was conducted in which the pulse period was varied between 1 ms or more and 1 s or less (duty ratio of 0.1 or more and 0.5 or less).

In the present test, perfluoropentane (boiling point 30° C.) was used as the low boiling-point, poorly water-soluble substance for phase change, and perfluoroheptane (boiling point 82° C.) was used as the high boiling-point, poorly water-soluble substance for phase change. However, the same effect was obtained when 1H-perfluorohexane (boiling point 70° C.) and perfluorooctane (boiling point 105° C.) were used as the high boiling-point, poorly water-soluble substance for phase change. Results substantially equivalent to those of the present test were also obtained when pentene (boiling point 30° C.) or pentane (boiling point 36° C.) was used as the low boiling-point, poorly water-soluble substance for phase change and hexene (boiling point 69° C.) or heptane (boiling point 98° C.) was used as the high boiling-point, poorly water-soluble substance for phase change.

### (Test 2) Test Concerning the Change in Reversibility Depending on the Carrier Composition

As in Test 1, the experiment system shown in FIG. 2 was used to examine the change in brightness upon irradiation of carriers having different components prepared by the following method with ultrasound. The below-indicated components were added together, and the mixture was homogenized with ULTRA-TURRAX T25 (Janke&Knukel, Staufen, Germany) at 9500 rpm for one minute at ice temperature while normal saline was slowly added until the total volume became 25 ml.

| | | |
|---|---|---|
| glycerol | 2.0 | g |
| α-tocopherol | 0.02 | g |
| cholesterol | 0.1 | g |
| lecithin | 1.0 | g |
| perfluoropentane | (A) | g |
| perfluoroheptane | (B) | g |

(A) and (B) are any of the following combinations (0:0.388, 0.0288:0.3492, 0.0576:0.3104, 0.0864:0.2716, 0.1152:0.2328, 0.144:0.194, 0.1728:0.1552, 0.2016:0.1164, 0.2304:0.0776, 0.2592:0.0388, and 0.288:0), which correspond to the molar ratios 0:10, 1:9, 2:8, 3:7, 4:6, 5:5, 6:4, 7:3, 8:2, 9:1, and 10:0, respectively.

This emulsion was subjected to high-pressure emulsification using Emulsiflex-C5 (Avestin, Ottawa, Canada) at 20 MPa for 2 minutes, and then filtered with a 0.4-μm membrane filter. These processes yielded substantially transparent microemulsion, of which 98% or more had diameters of 200 nm or smaller as measured with LB-550 (Horiba, Ltd., Tokyo).

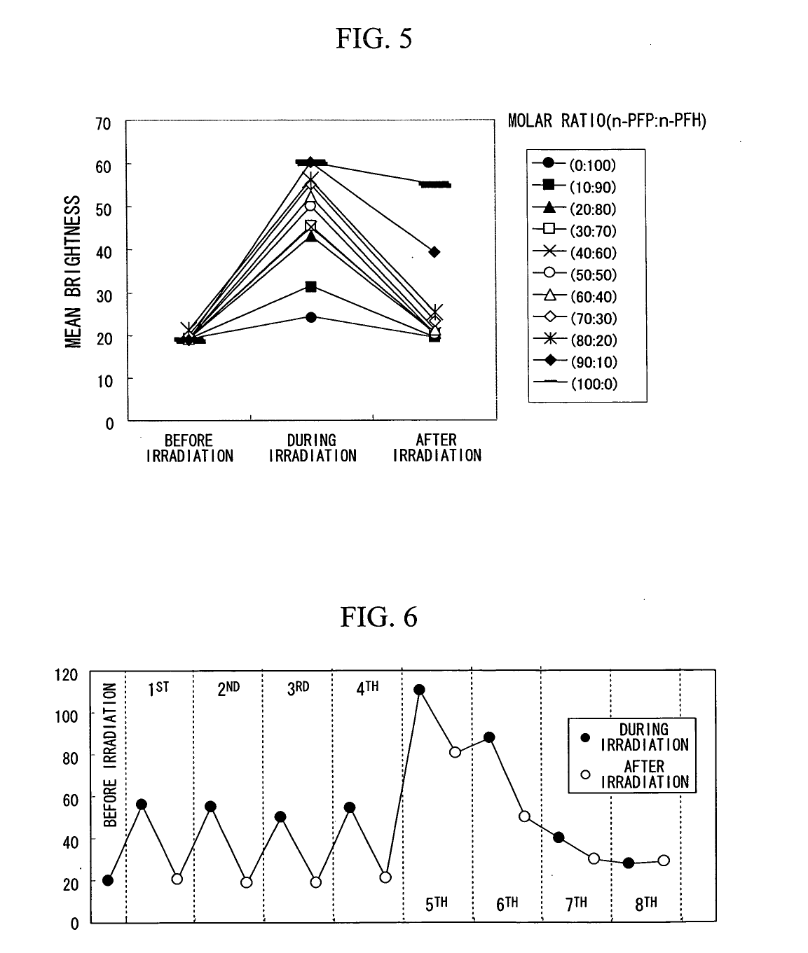

FIG. 5 shows a numerical representation of the mean brightness of ultrasound tomographic images of the sample that were obtained by the ultrasound diagnostic apparatus 9 in the WPI mode upon ultrasound irradiation by the transducer 7 for generating a focused ultrasound wave for sample-phase change at frequency 3.4 MHz, peak intensity 4 W/cm², and pulse period 35 ms (5 ms on, 30 ms off) for 0.5 second. From FIG. 5, it can be seen that an increase in brightness due to ultrasound irradiation is seen when the ratio of the low boiling-point, poorly water-soluble substance to the high boiling-point, poorly water-soluble substance was 10:90 or higher and 80:20 or lower, and that the brightness returns to levels substantially those prior to ultrasound irradiation upon termination of ultrasound. When the concentration of the low boiling-point, poorly water-soluble substance was 0%, there was little change in brightness due to ultrasound irradiation. The same results were obtained in a test in which the ultrasound peak intensity was varied in the range of 1 to 20 W/cm². Substantially the same results were obtained in a test in which the frequency of the ultrasound emitted by the transducer 7 for generating a focused ultrasound wave for sample-phase change was varied in the range of 0.5 to 7 MHz. The same was true in a test in which the pulse period was varied from 1 ms or greater and 1 s or smaller (duty ratio: 0.1 or more and 0.5 or less).

In the present test, perfluoropentane (boiling point 30° C.) was used as the low boiling-point, poorly water-soluble substance for phase change, and perfluoroheptane (boiling point 82° C.) was used as the high boiling-point, poorly water-soluble substance for phase change. However, the same effect was obtained when 1H-perfluorohexane (boiling point 70° C.) or perfluorooctane (boiling point 105° C.) was used as the high boiling-point, poorly water-soluble substance for phase change. Also, results substantially equivalent to those of the present test were obtained when pentene (boiling point 30° C.) or pentane (boiling point 36° C.) was used as the low boiling-point, poorly water-soluble substance for phase change, and hexene (boiling point 69° C.) or heptane (boiling point 98° C.) was used as the high boiling-point, poorly water-soluble substance for phase change.

### (Test 3) Test Concerning the Irreversible Change of the Carrier

After the carrier of the invention is encapsulated with a drug, the drug can be released by destroying the carrier at an appropriate timing. That such destruction of carrier (irreversible change) can be caused by ultrasound irradiation is demonstrated in a test described below. In the test, a carrier was prepared in the same way as in Test 1 and was tested using the experiment system shown in FIG. 2. An example of the results of the test is described with reference to FIG. 6.

FIG. 6 shows a numerical representation of the mean brightness of a sample during and 2 seconds after irradiation based on ultrasound tomographic images obtained by the ultrasound diagnostic apparatus 9 upon ultrasound irradiation by the transducer 7 at frequency 3.4 MHz, peak intensity 4 W/cm², and pulse period 35 ms (5 ms on, 30 ms off) for 0.5 second, which process was repeated four times, followed by ultrasound irradiation at frequency 3.4 MHz, peak intensity 100 W/cm², and pulse period 35 ms (10 ms on, 25 ms off) for 10 seconds.

In FIG. 6, during the initial four ultrasound irradiations, the brightness that had changed during ultrasound irradiation was back to original levels at the end of each ultrasound irradiation, indicating that the change in brightness is reversible. On the other hand, in the fifth irradiation, an increase in brightness of about twice the previous increase is seen during ultrasound irradiation, and the brightness decreases little following ultrasound irradiation. Thereafter, in the sixth and subsequent irradiations, no increase in brightness is seen during ultrasound irradiation, and the brightness decreases as the number of times of irradiation increases. This result indicates that, while in the first four ultrasound irradiations, a reversible phase-change between liquid and gas was seen, in the latter four irradiations including the fifth irradiation, the carrier is once turned gaseous and then destroyed, thus indicating the presence of an irreversible change. Thus, it is obvious that the drug carrier of the invention is capable of being irreversibly destroyed by ultrasound irradiation.

Substantially the same results were obtained when the frequency of the ultrasound emitted by the transducer 7 for generating a focused ultrasound wave for sample-phase change for causing irreversible destruction was varied in the range of 0.5 to 7 MHz. Substantially the same results were also obtained when an experiment was conducted using a pulsed or continuous wave having a pulse period of 1 ms or greater (duty ratio 0.1 or greater and 0.5 or smaller) and ultrasound intensity of 10 W/cm² or greater and 110 kW/cm² or smaller.

### EXAMPLE 1

An example of a drug carrier in which a lipophilic drug is encapsulated is described. The following components were added together and, while 20 ml of distilled water was slowly added, the mixture was homogenized with ULTRA-TURRAX T25 (Janke&Knukel, Staufen, Germany) at 9500 rpm at ice temperature for one minute.

| | | |
|---|---|---|
| glycerol | 2.0 | g |
| α-tocopherol | 0.02 | g |
| cholesterol | 0.1 | g |
| lecithin | 1.0 | g |
| perfluoropentane | 0.086 | g (300 nmol) |
| perfluoroheptane | 0.27 | g (700 nmol) |
| paclitaxel | 0.01 | g |

This emulsion was subjected to high-pressure emulsification using Emulsiflex-C5 (Avestin, Ottawa, Canada) at 20 MPa for 2 minutes, and then filtered by a 0.4-μm membrane filter. These processes yielded a substantially transparent microemulsion, of which 98% or more had diameters of 200 nm or smaller as measured with LB-550 (Horiba, Ltd., Tokyo). When it is desired to obtain emulsion greater than 200 nm for particular purposes, the high-pressure emulsification process may be omitted. The same results were obtained when 1 to 10% of the lecithin used was substituted by phosphatidylethanolamine to which PEG was added. The drug that is encapsulated is not particularly limited as long as it can be solubilized in the lecithin membrane. Thus, it was possible to encapsulate drugs other than paclitaxel, such as an anticancer drug such as adriamycin, or a lipophilic pigment sensitizing agent of porphyrins or xanthenes, by the same technique.

### EXAMPLE 2

An example of a drug carrier in which a water-soluble drug is encapsulated is described. In the present example, the water-soluble drug that is contained in the drug carrier is cisplatin. First, 0.01 g of an aqueous solution of cisplatin (0.1 mg/ml) was mixed with 0.2 ml of a soybean-oil solution of sorbitan sesquioleate (10 mg/ml), thereby forming a W/O emulsion (drug solution A). Thereafter, the following components were added together and, while 20 ml of distilled water was slowly added, the mixture was homogenized with ULTRA-TURRAX T25 (Janke&Knukel, Staufen, Germany) at 9500 rpm at ice temperature for one minute.

| | | |
|---|---|---|
| glycerol | 2.0 | g |
| α-tocopherol | 0.02 | g |
| cholesterol | 0.1 | g |
| lecithin | 1.0 | g |
| perfluoropentane | 0.086 | g |
| perfluorohexane | 0.24 | g |
| drug solution A | 0.1 | ml |

This emulsion was subjected to high-pressure emulsification using Emulsiflex-C5 (Avestin, Ottawa, Canada) at 20 MPa for 2 minutes, and then filtered by a 0.4-μm membrane filter. These processes yielded a substantially transparent microemulsion, of which 98% or more had diameters of 200 nm or smaller as measured with LB-550 (Horiba, Ltd., Tokyo). If an emulsion greater than 200 nm is required for particular purposes, the high-pressure emulsification process may be omitted. The same results were obtained when 1 to 10% of the lecithin used was substituted by phosphatidylethanolamine to which PEG was added. The surfactant for the preparation of drug solution A is not particularly limited as long as HLB is 5 or smaller. The drug is also not particularly limited as long as it can exist in the form of an aqueous solution.

### EXAMPLE 3

An example of a drug carrier in which a lipophilic drug dissolved in oil is encapsulated is described. The following components were added together, and, while normal saline was slowly added until the overall volume became 25 ml, the mixture was homogenized with ULTRA-TURRAX T25 (Janke&Knukel, Staufen, Germany) at 9500 rpm at ice temperature for one minute.

| | |
|---|---|
| glycerol | 2.0 g |
| α-tocopherol | 0.02 g |
| cholesterol | 0.1 g |
| lecithin | 2.0 g |
| perfluoropentane | 0.086 g |
| perfluorooctane | 0.28 g |
| soybean oil | 0.5 g |
| paclitaxel | 0.01 g |

This emulsion was subjected to high-pressure emulsification with Emulsiflex-C5 (Avestin, Ottawa, Canada) at 20 MPa for 2 minutes, and then filtered by a 0.4-μm membrane filter. These processes yielded a substantially transparent microemulsion, of which 98% or more had diameters of 200 nm or less as measured with LB-550 (Horiba, Ltd., Tokyo). If an emulsion greater than 200 nm is required for particular purposes, the high-pressure emulsification process may be omitted. The same results were obtained when 1 to 10% of the lecithin used was substituted by phosphatidylethanolamine to which PEG was added. The drug that is encapsulated is not particularly limited as long as it can be solubilized in the lecithin membrane. Thus, it was possible to encapsulate drugs other than paclitaxel, such as an anticancer drug such as adriamycin, or a lipophilic pigment sensitizing agent of porphyrins or xanthenes, by the same technique.

### EXAMPLE 4

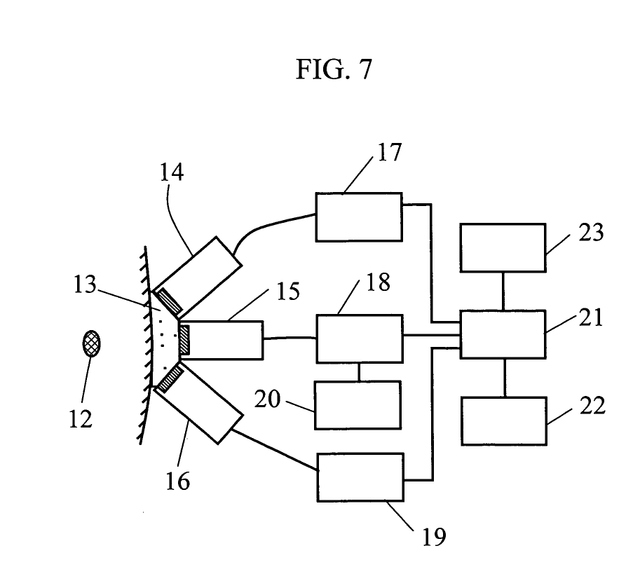

FIG. 7 is a diagram of an example of the ultrasound apparatus for releasing a drug according to the invention. The drug releasing device of the present example includes: a phase-changing ultrasound transmitting unit 14 disposed relative to a treatment subject 12 via an acoustic coupling material 13; a phase-change detecting ultrasound transmitting/receiving unit 15; a drug-releasing ultrasound transmitting unit 16; a phase-changing ultrasound control unit 17; a phase-change detecting ultrasound control unit 18; a drug-releasing ultrasound control unit 19; a phase-change determining signal processing unit 20; an integrated control unit 21; an image processing unit 22; and an input/display unit 23.

The phase-changing ultrasound transmitting unit 14 is capable of emitting ultrasound of either a single frequency selected from 0.5 to 10 MHz or a base frequency selected from 0.5 to 5 MHz and a frequency twice the base frequency, the ultrasound of each frequency having an acoustic intensity of 0.5 to 10 W/cm². The phase-change detecting ultrasound transmitting/receiving unit 15 is capable of transmitting and receiving ultrasound of frequencies that can be used in typical ultrasound diagnostic apparatuses, i.e., on the order of roughly 2 to 10 MHz, and having an acoustic intensity of not more than 0.72 W/cm² in temporal mean intensity. The drug-releasing ultrasound transmitting unit 16 is capable of emitting ultrasound of either a single frequency selected from 0.5 to 10 MHz, or a base frequency selected from 0.5 to 5 MHz and a frequency twice the base frequency, having any acoustic intensity value selected from the range of 10 to 10 kW/cm². The drug-releasing ultrasound transmitting unit 16 may also be used for therapeutic ultrasound irradiation.

The integrated control unit 21 is operated in any of the following modes: a mode in which it operates the phase-changing ultrasound transmitting unit 14 by controlling the phase-changing ultrasound control unit 17; a mode in which it operates the phase-change detecting ultrasound transmitting/receiving unit 15 by controlling the phase-change detecting ultrasound control unit 18; and a mode in which it operates the drug-releasing ultrasound transmitting unit 16 by controlling the drug-releasing ultrasound control unit 19. The mode in which the phase-change detecting ultrasound control unit 18 is controlled to operate the phase-change detecting ultrasound transmitting/receiving unit 15 is carried out immediately following the mode in which the phase-changing ultrasound control unit 17 is controlled to operate the phase-changing ultrasound transmitting unit 14. The phase-changing ultrasound transmitting unit 14 and the phase-change detecting ultrasound transmitting/receiving unit 15 may share a single ultrasound transducer. Preferably, the drug-releasing ultrasound transmitting unit 16 employs a dedicated ultrasound transducer.

The phase-change determining signal processing unit 20 is capable of image processing for the quantification of changes in the intensity or frequency components of an ultrasound echo signal produced by a phase-change in the contrast agent. For the quantification, a before-phase-change signal recording unit and an after-phase-change signal recording unit may be used. The former is used for storing an ultrasound echo signal prior to phase-changing ultrasound irradiation. The latter is used for storing an ultrasound echo signal during or after the phase-change ultrasound irradiation. The difference between these stored signals in terms of specific frequency components may be determined by a computation unit. Particularly, it is desirable to compare the even harmonics components of the central frequencies of the phase-change detecting ultrasound before and during or after the phase-changing ultrasound irradiation.

The apparatus may be configured such that ultrasound irradiation by the drug-releasing ultrasound transmitting unit 16 is permitted only after confirming the presence of the phase-change type contrast agent at the affected area 12 based on image processing by the phase-change determining signal processing unit 20, upon detection by the phase-change detecting ultrasound transmitting/receiving unit 15 of a phase change in the contrast agent at the affected area 12 caused by ultrasound irradiation by the phase-changing ultrasound transmitting unit 14. For example, if the apparatus is turned on and an action is taken to activate the drug-releasing ultrasound transmitting unit 16 to carry out ultrasound irradiation without conducting ultrasound irradiation with the phase-changing ultrasound transmitting unit 14, an alert may be issued to prompt the user to implement ultrasound irradiation using the phase-changing ultrasound transmitting unit 14. Alternatively, after ultrasound irradiation by the phase-changing ultrasound transmitting unit 14, an image may be acquired through the transmission and reception of ultrasound by the phase-change detecting ultrasound transmitting/receiving unit 15, and then the drug-releasing ultrasound transmitting unit 16 may be controlled to carry out ultrasound irradiation at a region where a phase change in the phase-change type ultrasound contrast agent has been identified through the reception of an ultrasound echo signal having an intensity exceeding a predetermined level.

In accordance with the drug releasing device of the present example, the presence of drug can be identified without spilling it, thus making it possible to release the drug after confirming that it is properly accumulated at the target site. Thus, diagnosis and therapy can be conducted safely.

What is claimed is:

1. A drug carrier comprising:
a mixture of a first poorly water-soluble compound having a boiling point below the body temperature of a subject of administration of a drug, and a second poorly water-soluble compound having a boiling point exceeding the body temperature of the subject; and
a drug contained in the mixture,
wherein the mixture is encased in a membrane made of an amphipathic substance.

2. The drug carrier according to claim 1, wherein the body temperature is 37° C., and the first poorly water-soluble compound and the second poorly water-soluble compound have a molar ratio of 10:90 or greater and 80:20 or smaller.

3. The drug carrier according to claim 1, wherein the first poorly water-soluble compound is vaporized by ultrasound irradiation, and the second poorly water-soluble compound is secondarily vaporized by the ultrasound absorption by the vaporized first poorly water-soluble compound.

4. The drug carrier according to claiin 1, wherein the mixture is liquid when administered and rendered gaseous upon ultrasound pulse irradiation with a peak intensity of 1 to 20 W/cm², the mixture returning to the original liquid upon termination of ultrasound irradiation.

5. The drug carrier according to claim 1, wherein the second poorly water-soluble compound has a structure such that at least one hydrogen atom or halogen atom of the first poorly water-soluble compound is substituted with an alkyl group or an alkyl halide group.

6. The drug carrier according to claim 1, wherein the second poorly water-soluble compound has a structure such that at least one halogen atom of the first poorly water-soluble compound is substituted with a hydrogen atom.

7. The drug carrier according to claim 1, wherein the drug is water-soluble.

8. The drug carrier according to claim 1, wherein the drug is lipophilic.

9. An ultrasound apparatus comprising:
an ultrasound transducer for transmitting and receiving ultrasound to and from a subject;
a control unit for controlling the ultrasound transducer; and
an image generating unit for generating an image based on a signal received by the ultrasound transducer,
wherein the control unit causes the ultrasound transducer to be operated in a first mode in which the transducer emits a ultrasound pulse having a peak intensity of 1 to 20 W/cm², a second mode in which a ultrasound image of the subject is obtained, and a third mode in which the transducer emits an ultrasound pulse having a peak intensity of 10 to 10 kW/cm².

10. The ultrasound apparatus according to claim 9, comprising a plurality of ultrasound transducers, wherein one of the ultrasound transducers is a dedicated ultrasound transducer for the third mode.

11. The ultrasound apparatus according to claim 9, wherein operation in the third mode is permitted on the condition that operation in the first mode has been carried out.

12. The ultrasound apparatus according to claim 10, wherein a region of an ultrasound image obtained in the second mode immediately after the first mode in which the brightness exceeds a predetermined level is irradiated with an ultrasound pulse in the third mode.
